(19) United States
(12) Patent Application Publication
(10) Pub. No.: US 2015/0059739 A1
(43) Pub. Date: Mar. 5, 2015

(54) INHALER SPACER

(71) Applicant: CENTRAL MANCHESTER UNIVERSITY HOSPITALS NHS FOUNDATION TRUST, Manchester Lancashire (GB)

(72) Inventor: Tariq Aslam, Manchester Greater Manchester (GB)

(21) Appl. No.: 14/379,856

(22) PCT Filed: Feb. 12, 2013
(86) PCT No.: PCT/GB2013/050320

§ 371 Date: Aug. 20, 2014

(30) Foreign Application Priority Data
Feb. 21, 2012 (GB) 1202926.0

(51) Int. Cl. A61M 15/00

(52) U.S. Cl. CPC A61M 15/0086; A61M 15/0016; A61M 15/0021; A61M 15/0065; A61M 2205/3331; A61M 2205/52; A61M 2205/505; A61M 2205/18
USPC 128/202.22; 128/203.12

(57) ABSTRACT

A device for use with an inhaler spacer to improve compliance with a medicament dosage regime. The device comprises a display, detection means for detecting indications of successful inhalations by a patient using an inhaler spacer and a memory storing computer readable instructions. The computer readable instructions are configured to present at least one incentive graphic on said display, and to update the incentive graphic in response to detection of an indication of a successful inhalation by the detection means.

The present invention is concerned with methods and systems for improving a user's compliance with a medicament dosage regime requiring inhalation of a predetermined dosage of a medicament using an inhaler spacer. The methods and systems are particularly, but not exclusively, applicable for use with children.

Inhaled medicaments are commonly prescribed to patients for treating diseases such as asthma and viral induced wheeze. Inhaled medicaments may be administered via a number of means, including metered dose inhalers (MDIs) and nebulisers. MDIs are the most commonly prescribed means for administering inhaled medicaments.

In order to properly use an inhaler, a number of coordinated actions are required (pressing down on the inhaler, breathing in deeply as the medication is released, holding your breath and exhaling). Some patients, for example the very young or elderly, have difficulty completing the required actions in the correct order and at the correct times.

Patients who experience difficulty using an MDI are commonly provided with a spacer device. Spacer devices connect a drug delivery canister (for example, an MDI) to a mask or mouthpiece. Pressing of the drug canister releases the drug into a chamber of the spacer device. The medicament is held within the chamber by a valve, which is opened upon inhalation, allowing the patient to inhale the treatment in his own time through the mask. Cessation of an inhalation causes closure of the valve to keep the remaining drug in the chamber of the spacer device.

Use of the spacer device avoids timing issues experienced with MDIs. Indeed, a patient is generally encouraged to breath “normally” when using a spacer device, rather than to adopt a particular, abnormal, breathing pattern. Further, aerosol is generally issued to the patient from the spacer device more slowly than when issued directly from the MDI, resulting in less of the drug impacting on the back of the mouth and more of the drug reaching the lungs of the patient. Because of this, less medication is needed for an effective dose to reach the lungs, and there are fewer negative side effects, for example from corticosteroid residue in the mouth.

For children, spacer devices are particularly useful, allowing them to gain the benefit of inhaled drugs in a way that they can use at home, without the use of hospital nebulisers and masks. In fact, studies have shown that used correctly, these devices can match the efficacy of hospital nebulisers in treatment of asthmatic children.

Spacer devices, do however, suffer from a number of problems which can limit their effectiveness. For example, in order to use a spacer device correctly, the patient is required to make an effective seal with the mask. A poor seal will not allow efficient drug delivery. The quality of the seal may be assessed by a parent/supervisor watching a valve of the spacer device to ensure that the valve is moving and by listening for an accompanying sound. The valves, however, are small, such that assessing the quality of the seal this way is often difficult, especially at night.

Further, once a correct seal is made, the patient is required to take multiple separate breaths to inhale the drug. This process may itself need to be repeated multiple times and for severe attacks may need to be performed hourly or two hourly. The difficulty faced by some patients is therefore increased just at the moment when an effective treatment is most in need. For children, such repetition often leads boredom, distraction and an unwillingness to participate, especially when tired or unwell. Such disadvantages can hinder efforts to encourage a patient to adopt their normal breathing pattern, which is required to achieve the most effective administration of the medicament.

It is an object of the present invention to obviate or mitigate one or more of the problems outlined above.

According to a first aspect of the present invention, there is provided a device for use with an inhaler spacer to improve compliance with a medicament dosage regime, the device comprising: a display; detection means for detecting indications of successful inhalations by a patient using an inhaler spacer; a memory storing computer readable instructions configured to: present at least one incentive graphic on said display; and to update said incentive graphic in response to detection of an indication of a successful inhalation by said detection means.

By presenting an incentive graphic to a user of an inhaler spacer, and updating the incentive graphic in response to detection of an indication of successful inhalation, the user of the inhaler spacer is provided with an adaptable and interactive visual indication of each correct inhalation. The user is therefore motivated to continue correct usage, and to correct incorrect usage of the inhaler spacer.

The indications of successful inhalations may comprise indications of successful inhalations of a medicament. The indications of successful inhalations may comprise exhalations through an outlet of said inhaler spacer. In this way, the indications of successful inhalation are more easily measured than indications which rely upon directly detecting inhalations. The indications of successful inhalations may comprise exhalations having at least a predetermined pressure at the detection means. By only accepting inhalations with at least a predetermined pressure, more certainty is provided that the detected exhalation corresponds to a successful inhalation.

The detection means may comprise a microphone, thereby providing a simple, efficient and cost effective way to detect indications of successful inhalations.

The device may further comprise mounting means adapted to mount the display and the detection means to an inhaler spacer. In this way, backwards compatibility is provided with standard inhaler spacers, thereby further increasing the cost effectiveness of the device.

The computer readable instructions may be further configured to receive data indicating patient details and to configure the displayed graphic in response to the received data. For example, the computer readable instructions may be configured to receive details regarding the patient's age, and how much of a medicament they are required to inhale. In this way, the incentive graphic can be adapted automatically so that it is appropriate to the particular patient details received.

The graphic may be provided as part of a game, wherein the game is configured based upon the received data. For example, the received data may indicate a required number of cycles (i.e. times that a patient must repeat a particular number of inhalations) and the game may be configured to provide a number of levels equal to the required number of cycles. In this way, the number of levels provided by the game matches the number of cycles that the patient must complete in order to complete the dosage regime.

The received data may indicate a required number of inhalations per cycle and the game may be configured to provide a number of stages for each level equal to the required number of inhalations. In this way, where a patient needs to take a particular number of breaths in each cycle, the number of stages in each level of a provided game may match the number of breaths.

The incentive graphic may be adapted to indicate progress towards completion of the dosage regime. For example, the incentive graphic may display a endpoint of the game, at which point the patient will have completed either all or a part of the dosage regime. The patient may then be able to see their progress towards that endpoint, thereby providing further motivation. For example, where the incentive graphic is provided as part of a game with a plurality of levels, the user may be able to see which level they are currently at, and how many levels are remaining until completion of the game. Further, where each level provides a number of stages corresponding to a required number of inhalations, the user may be able to see how many of the required number of stages have been completed, thereby providing further motivation to complete each inhalation.

The memory may comprise a plurality of games and the computer readable instructions may be configured to select one of the plurality of games based on received details.

Where the received data comprises data indicating an age of the patient, a difficulty level of the game may be configured based upon the indicated age. For example, younger patients may be presented with games with conceptually simple objectives, such as blowing away balloons. Older patients may be presented with more conceptually complex games such as blowing a man's boat across a river. A series of games may be linked by a storyline, which may be configured based upon the received data.

The computer readable instructions may be further configured to, if an indication of a successful inhalation is not detected by the detection means within a predetermined time period, cause a prompt to be presented on the display to incentivise the user to complete a successful inhalation of the medicament.

The detection means may be movable in relation to the outlet, and the computer readable instructions may be configured to perform a calibration operation comprising: receiving from said detection means a first calibration pressure; determining whether the first calibration pressure satisfies a predetermined criterion; and displaying a prompt on the display for the detection means to be moved in relation to the outlet if the first calibration pressure does not satisfy said predetermined criterion. In this way, the device can be configured for use with different patients and for different requirements.

According to a second aspect of the present invention, there is provided a computer implemented method for improving compliance with a medicament dosage regime, said regime requiring inhalation of a predetermined dosage of a medicament using an inhaler spacer, the method comprising: displaying at least one incentive graphic on a display device; detecting a successful inhalation of said medicament; and in response to detecting said successful inhalation, updating said incentive graphic.

According to a third aspect of the present invention, there is provided a device mount for use with an inhaler spacer, comprising: support means configured to support a device according to the first aspect.

According to a fourth aspect of the present invention, there is provided an inhaler spacer device, comprising: a mouthpiece; a spacer configured to connect the mouthpiece to a medicament delivery device, said spacer having an air outlet; and an incentive device comprising: detection means for detecting indications of a successful inhalation by a user of the inhaler device; a display; and a memory storing computer readable instructions configured to receive indications of successful inhalations detected by said detection means and to provide an incentive graphic on said display, said incentive graphic being updated in response to received indications of successful inhalations to indicate progress towards completion of a predetermined dosage requirement.

According to a fifth aspect of the present invention, there is provided a device for use with an inhaler, the device comprising: a display; detection means for detecting indications of successful inhalations by a patient; a memory storing computer readable instructions configured to: present at least one incentive graphic on said display; and to update said incentive graphic in response to detection of an indication of a successful inhalation by said detection means.

According to a sixth aspect of the present invention, there is provided a kit of parts for assembling an inhaler spacer for improving compliance with a medicament dosage regime, comprising a mouthpiece; a spacer configured to connect the mouthpiece to a medicament delivery device, said spacer having an air outlet; and an incentive device comprising: detection means for detecting indications of a successful inhalation by a user of the inhaler spacer; a display; and a memory storing computer readable instructions configured to receive indications of successful inhalations detected by said detection means and to provide an incentive graphic on said display, said incentive graphic being updated in response to received indications of successful inhalations to indicate progress towards completion of a predetermined dosage requirement.

According to a seventh aspect of the present invention, there is provided a kit of parts to be assembled to provide a device for use with an inhaler spacer to improve compliance with a medicament dosage regime, the kit comprising: a display; detection means for detecting indications of successful inhalations by a patient using an inhaler spacer; a memory storing computer readable instructions configured to: present at least one incentive graphic on said display; and to update said incentive graphic in response to detection of an indication of a successful inhalation by said detection means.

According to a eighth aspect of the present invention, there is provided a peak flow meter comprising: an inhaler spacer device comprising a mouthpiece and a spacer having an air outlet; an electronic device comprising detection means for detecting indications of exhalations of a user of the inhaler spacer device; a display; and a memory storing computer readable instructions configured to receive indications of exhalations detected by said detection means; to provide an incentive graphic on said display, said incentive graphic being updated in response to received indications of exhalations; and to determine a peak expiratory flow rate based on said received indication.

It will be appreciated that aspects of the present invention can be implemented in any convenient way including by way of suitable hardware and/or software. For example, a dedicated hardware device may be provided, or a programmable device may be programmed to implement embodiments of the invention. For example, embodiments of the present invention may be implemented on mobile devices such as “smartphones” or tablet computers. The invention therefore also provides suitable computer programs for implementing aspects of the invention. Such computer programs can be carried on suitable carrier media including tangible carrier media (e.g. hard disks, CD ROMs and so on) and intangible carrier media such as communications signals.

It will be appreciated that features presented in the context of one aspect of the invention in the preceding and following description can equally be applied to other aspects of the invention.

Embodiments of the present invention are now described by way of example, with reference to the accompanying drawings, in which:

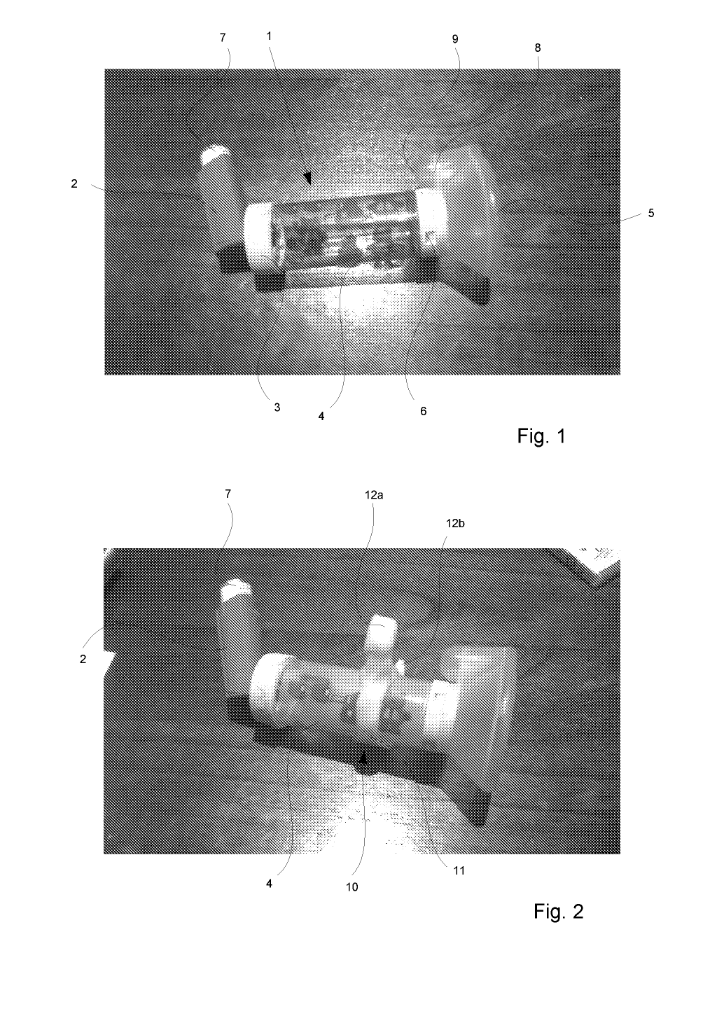

FIG. 1 is an image of a spacer device connected to a metered dose inhaler;

FIG. 2 is an image of a metered dose inhaler to which a device mount has been fixed in accordance with an embodiment of the present invention;

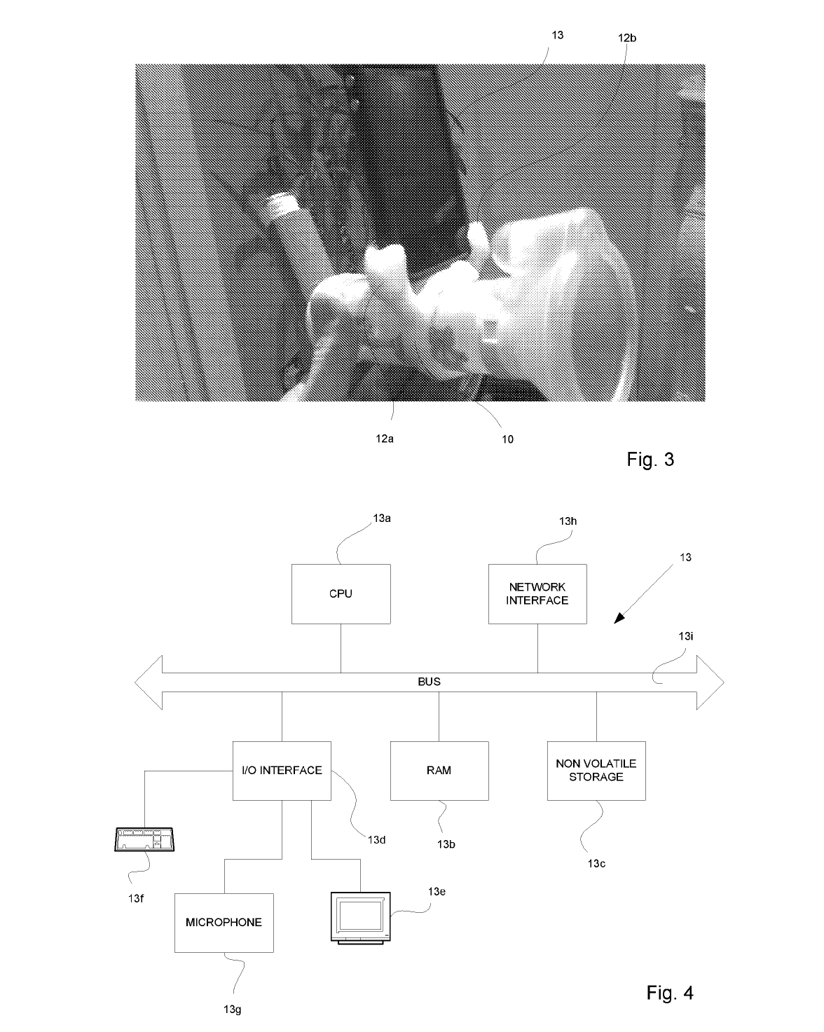

FIG. 3 is an image of a device mounted in the device mount in accordance with embodiments of the present invention;

FIG. 4 is a schematic illustration of example components of the device mounted in the device mount of FIG. 3;

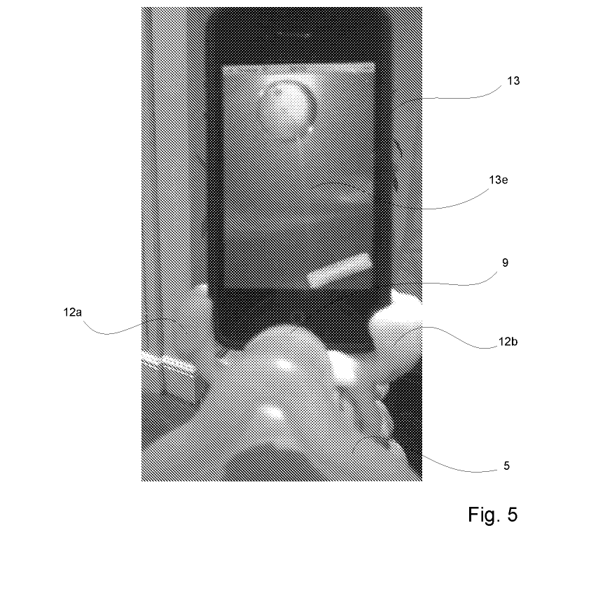

FIG. 5 is an image of the device mounted in the device mount of FIG. 3 as viewed in use by a user of the spacer device;

FIG. 6 is a flowchart showing a calibration process carried out by the device in accordance with an embodiment of the present invention;

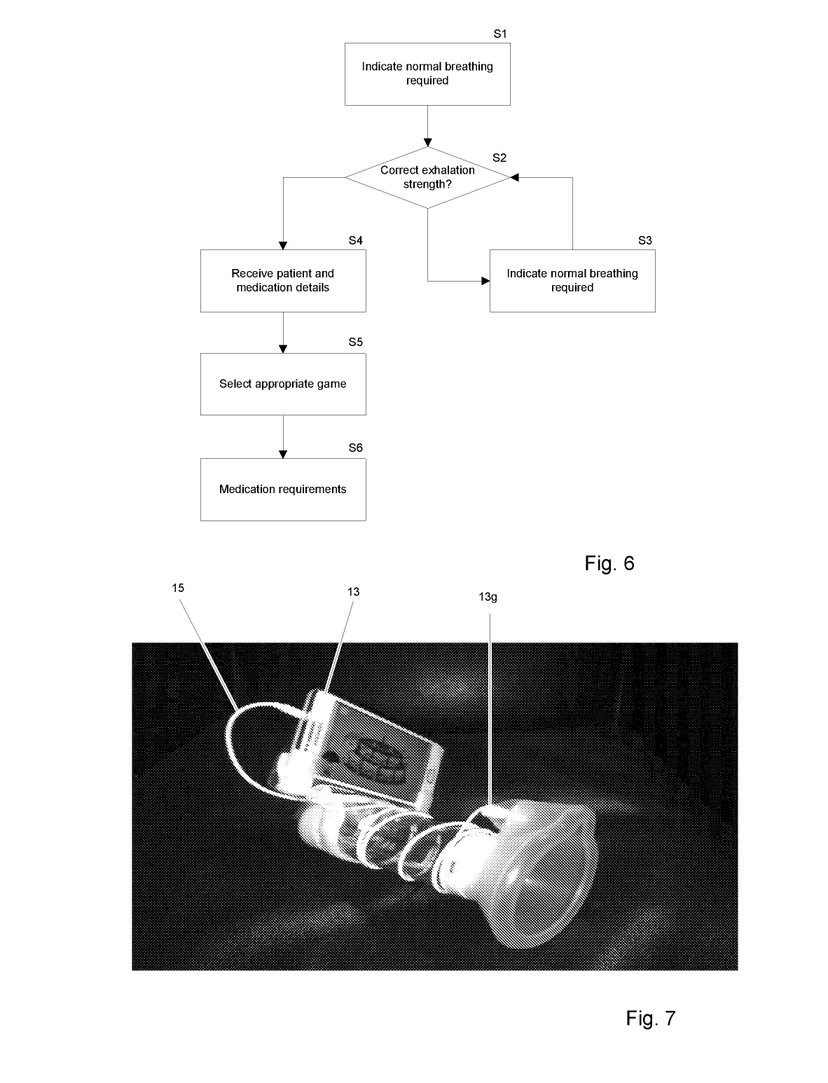

FIG. 7 is an image of the device mounted in the device mount in which a microphone is connected to the device by a wire; and

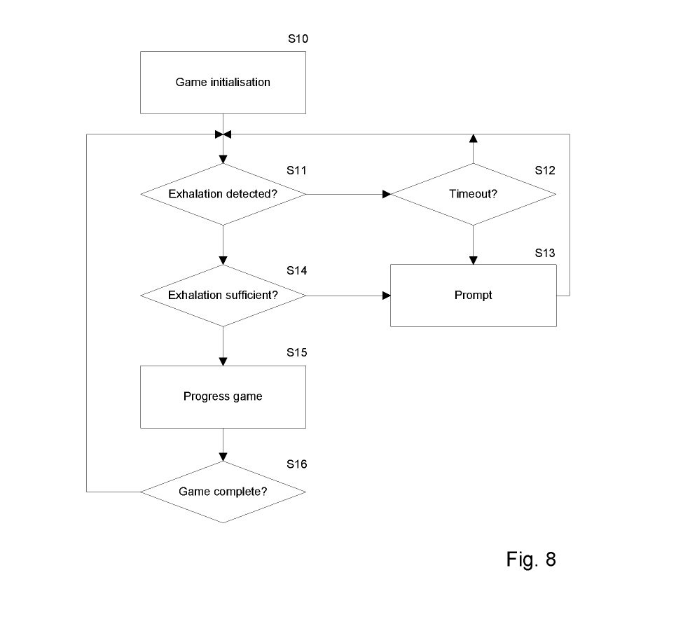

FIG. 8 is a flowchart showing processing carried out by the device to display a game to a patient in accordance with an embodiment of the present invention.

Referring to FIG. 1, a spacer device 1 is connected to a metered dose inhaler (MDI) 2 by way of a connector 3. The spacer device 1 further comprises a chamber 4 defined by a hollow cylindrical body connected at one end to the connector 3 and connected at an opposite end to a mask 5 by way of a connector 6. The chamber 4 is closed at one end by the MDI 2 and at an opposite end by an inlet valve (not visible) between the chamber 4 and the mask 5.

In use, a patient depresses a drug container 7 protruding from the MDI 2 to release a drug contained within the drug container 7 into the chamber 4. The inlet valve between the cylindrical chamber 4 and the mask 5 holds the released drug in the chamber 4. Upon proper fitment of the mask 5 to the patient's face, inhalation of the patient causes release of the inlet valve, allowing a portion of the drug within the chamber 4 to be inhaled by the patient. At the end of the patient's inhalation, the inlet valve closes such that any remaining drug is kept within the chamber 4. Exhalation of the patient opens a valve 8 in the mask 5 causing the patient's exhaled breath to be expelled through an outlet 9 of the mask 5. Repeated inhalation/exhalation cycles can be performed until all of the drug contained within the chamber 4 has been inhaled by the patient.

Referring to FIGS. 2 and 3, a mount 10 is connected to the cylindrical body of the spacer device 1. The mount 10 comprises a ring portion 11 adapted for sliding engagement around the cylindrical body of the spacer device 1. Support arms 12a, 12b extend from the ring portion 11, the support arms 12a, 12b being configured to hold an electronic device 13 (as shown in FIG. 3). The mount 10 fits tightly around the cylindrical body such that unintentional movement of the mount 10 relative to the cylindrical body is avoided. The mount 10 may, however, be moved along the cylindrical body to configure the distance between the electronic device 13 and the outlet 9 of the mask 5. The mount 10 may be locked into a position along the cylindrical body at a suitable distance from the outlet 9. This provides flexibility to adapt the configuration of the spacer device 1 and the mount 10 to the needs of particular patients and to particular objectives. In use the mount 10 is arranged to allow alignment of the outlet 9 with a microphone of the electronic device 13. The mount 10 is removable from the spacer device 1 to allow the mount 10 to be sterilised after use. For consistency, a scale (not shown) may be provided on the cylindrical portion 4, to allow a patient, or a supervisor of a patient to repeatedly select a particular distance for the mount 10 on the scale.

In general terms, interactive games, adapted to encourage a patient to inhale a required number of breaths, are displayed on a screen of the electronic device 13, the games being controlled by a patient's exhalations. In more detail, games operating on the electronic device are configured to detect the patient's exhalations and in response to each detected exhalation, to manipulate aspects of the games in a way that will keep the patient motivated and entertained throughout the proposed period of treatment, and which will encourage the patient to adopt a normal breathing pattern.

An example of components of the electronic device 13, which can be used to implement embodiments of the present invention, is now described with reference to FIG. 4. It will be appreciated that the components of the electronic device 13 illustrated in FIG. 4 and described below are merely exemplary, and that any suitable electronic device may be used to implement embodiments of the present invention. The electronic device 13 comprises a CPU 13a which is configured to read and execute instructions stored in a volatile memory 13b which takes the form of a random access memory. The volatile memory 13b stores instructions for execution by the CPU 13a and data used by those instructions. In the present embodiment, the instructions stored in the volatile memory 13b are instructions to cause the CPU 13a to run an application providing interactive games designed to encourage a patient to properly inhale a required amount of a drug.

The electronic device 13 further comprises non-volatile storage 13c. The non-volatile memory may take any appropriate form, such as a solid state drive (SSD), or a hard disk drive (HDD). The electronic device 13 further comprises an I/O interface 13d to which are connected input and output devices used in connection with the electronic device 13. More particularly, a display 13e is configured so as to display output from the electronic device 13. The display 13e displays the visual outputs of the interactive game during use. Input devices connected to the I/O interface 13d may include physical keys 13f which allow user interaction with the electronic device 13. Alternatively or additionally, the display 13e may provide a touchscreen allowing a patient or supervisor to interact with a user interface displayed on the touchscreen. A microphone 13g is also connected to the I/O interface 13d allowing sound input to be provided to the electronic device 13. In particular, the microphone 13g allows a patient's exhalations to be detected by the electronic device 13 for processing by the application. A network interface 13h allows the electronic device 13 to be connected to an appropriate computer network so as to receive and transmit data from and to other computing devices. In this way, the electronic device 13 may receive updates (for example, new games), or receive/transmit patient details/progress (e.g. readings from use of the device) from/to an external computing device (of, for example, a clinician). The CPU 13a, volatile memory 13b, non-volatile storage 13c, I/O interface 13d, and network interface 13h, are connected together by a bus 13i.

As described above, the device 13 runs an application configured to detect signals from the microphone 13g indicating pressure from an exhalation. The detected signals are used as input for games that can be played by the patient. Each game is represented on the display 13e, which is positioned directly in patient's line of sight (as can be seen in FIG. 5). In the example illustrated in FIG. 5, the application running on the device 13 is providing a game in which each detected exhalation causes a bubble, shown on the screen 13e, to inflate and float away.

The device 13 and mask 5 may be configured such that exhalation causes sufficient pressure on the microphone 13g to generate a signal only if a proper seal is made with the mask 5. In this way, the patient is required to maintain a proper seal with the mask 5 in order to operate the game. Unintentional failure to manipulate the game objects displayed on the screen 13e therefore act as an efficient indicator of incorrect usage of the spacer 1, and can provide motivation to correct usage.

A calibration process performed by the device 13 is now described with reference to FIG. 6. At a step S1 the application displays a prompt on the screen 13e indicating that the patient should breathe normally into the mask 5. At this stage, a parent or supervisor may be required to ensure that correct breathing is maintained and that a proper seal has been made with the mask 5. At a step S2, the application processes signals from the microphone 13g to determine whether a acceptable exhalation pressure is detected at a current distance of the microphone 13g from the outlet 9. If it is determined that an acceptable exhalation pressure is not detected at the microphone 13g, processing passes to a step S3 at which a prompt is displayed on the screen 13e for the patient or supervisor to alter the distance of the mount 10 from the mask 5. In particular, if excessive pressure is detected at the microphone 13g, a prompt is displayed indicating that the mount 10 (and therefore the microphone 13g) is to be slid along the cylindrical portion 4 away from the mask 5. On the other hand, if insufficient, or no, pressure is detected at the microphone 13g, a prompt is displayed indicating that the mount should be slid towards the mask 5. Processing passes from step S3 back to step S2 and continues to loop between steps S2 and S3 until a correct pressure is detected, indicating that the microphone 13g is correctly positioned with respect to the outlet 9 of the mask 5.

If a correct exhalation strength is detected at the microphone 13g, processing passes from step S2 to a step S4 at which the screen 13e displays a prompt for the patient or supervisor to enter the patient's age and how many depressions of the drug canister are required. In general, each depression of the canister requires ten inhalations/exhalations by the patient, although it will be appreciated that this may vary in dependence upon a number of factors including, for example, the size of the inhaler spacer. Upon receiving the patient and dosage details, processing passes to a step S5 at which the application uses the details received at step S4 to select and configure an appropriate game. For example, where a patient requires 10 cycles of 10 inhalations, a selected game may be configured to provide 10 levels, each level comprising 10 stages before completion of that level. A difficulty level of the game may be selected depending upon an age of the patient.

In the above described calibration process, the device 13 is slid along the cylindrical portion 4 to adapt the distance between the outlet 9 and the microphone 13g. Referring to FIG. 7, in other embodiments of the present invention, the microphone 13g may be attached directly to the outlet 9 and connected to the device via a wire 15. In such embodiments, calibration may be performed through software operating on the device 13 rather than by movement of the microphone 13g with respect to the outlet 9. In particular, a sensitivity of the microphone 13g may be calibrated using software operating on the device 13. Further, it will be appreciated that calibration may not be necessary for all patients.

As described above, the games presented to the patient are configured to respond to exhalation ejected from the outlet 9 to trigger a response on the screen or similarly gain the attention and compliance of the patient. It will be appreciated that a wide variety of games may be provided and that the provided games can take any appropriate form. Merely as examples, games may comprise blowing of bubbles to win a competition, blowing out candles on a cake, blowing leaves off a tree, etc. Each game may be stored on the device 13 (i.e. in the non-volatile storage 13c) with corresponding meta-data indicating a suitable age range, possible number of levels, number of stages per level etc, the meta-data being used to select appropriate games based upon the patient details received at step S4.

Processing carried out by the device 13 to present a game is now described with reference to FIG. 8. At a step S10 the game selected at step S5 of FIG. 6 is initialised. Processing passes from step S10 to a step S11 at which it is determined whether an exhalation has been detected at the microphone 13g. If it is determined that an exhalation has not been detected, processing passes to a step S12 at which it is determined whether a timeout condition has occurred. If, at step S12 it is determined that a timeout condition has not occurred, processing passes back to step S11. If, on the other hand, it is determined that a timeout condition has occurred, processing passes to a step S13 at which a motivational prompt is displayed on the screen to encourage the patient, or a supervisor, to ensure that the inhaler is being used correctly. For example, a prompt may be displayed to request that the supervisor ensure that a correct seal has been made with the mask 5. Processing passes from step S13 back to step S11.

If, at step S11, an exhalation is detected at the microphone 13g, processing passes to step S14, at which it is determined whether the detected exhalation is sufficiently strong to indicate correct usage of the inhaler (e.g. whether a correct seal has been made with the mask 5). If, at step S14, it is determined that the detected exhalation was not sufficiently strong, processing passes to step S13. Processing loops between steps S11, S12, S13 and S14 until a sufficiently strong exhalation is detected at the microphone 13g. Upon detection of a sufficiently strong exhalation at step S14, processing passes to a step S15 at which the patient's progress in the game is updated to indicate the successful inhalation/exhalation. The updated progress in the game causes a corresponding updating of game graphics displayed on the screen 13e to display the patient's progress and motivate further progress. The game may display an indication of an end point of the game so that the patient can easily see their progress towards the endpoint of the game, and therefore their progress towards inhaling the required dosage.

As described above with reference to FIG. 8, a patient's progress in the game is updated in response to detection of successful inhalation/exhalation. In some embodiments of the present invention, detected exhalations which are not sufficiently strong to indicate a fully successful inhalation/exhalation also produce an effect on the screen 13e to encourage more persistent/greater strength of exhalation. For example, in a game to blow out candles on a cake (where a candle being blown out indicates a fully successful inhalation/exhalation), candles may be caused to flicker in response to a detected exhalation which is not sufficiently strong to constitute a successful inhalation/exhalation. A further example may be in a game to blow bubbles, a bubble may be shown to partially inflate, but to subsequently deflate.

In some embodiments of the present invention, the device 13 may be adapted to provide an alarm to remind the patient to use the inhaler at predetermined times.

The arrangement of FIG. 2 may further be used as a peak flow meter. Peak flow is a measure of a person's maximum speed of respiration. Home monitoring of a patient's peak flow, with a peak flow meter, can help diagnose conditions such asthma. When used as a peak flow meter, a constant distance is maintained between the microphone 13g and the outlet 9 of the mask 5 in order to gather comparable intra-patient measurements. Games run on the device 13 and displayed on the screen 13e are configured to encourage a patient to momentarily exhale as forcefully as possible (e.g. to blow out a house on fire). The measured exhalation is then used to determine a peak expiratory flow rate based upon a relationship between detected exhalations and peak expiratory flow rates for patients based on clinical data (e.g. age, gender, etc). For example, rises in noise levels detected by the microphone over a short period of time may correspond with strength of exhalation in peak flow.

It will be appreciated that while the above description describes use of a microphone 13g for detecting exhalations of a patient, any suitable exhalation detection means may be used (for example a pressure sensor).

While the above description has been concerned with the display of electronic “games” to a patient, it will be appreciated that any content may be displayed on the screen 13e which is suitable for motivating a user to complete a required number of inhalations of a drug. For example, a moving scene may be displayed, wherein the patient must complete regular, successful inhalations/exhalations in order to maintain the movement of the scene.

Further modifications and applications of the present invention will be readily apparent to the appropriately skilled person from the teaching herein, without departing from the scope of the appended claims.

1-23. (canceled)

24. A device for use with an inhaler spacer to improve compliance with a medicament dosage regime, the device comprising:
a display;
detection means for detecting indications of successful inhalations by a patient using an inhaler spacer;
a memory storing computer readable instructions configured to:
present at least one incentive graphic on said display; and
to update said incentive graphic in response to detection of an indication of a successful inhalation by said detection means.

25. A device according to claim 24, wherein said indications of successful inhalations comprise exhalations through an outlet of said inhaler spacer.

26. A device according to claim 24, wherein said indications of successful inhalations comprise exhalations having at least a predetermined pressure at said detection means.

27. A device according to claim 24, wherein said detection means comprises a microphone.

28. A device according to claim 24, further comprising mounting means adapted to mount said display and said detection means to an inhaler spacer.

29. A device according to claim 24, wherein said computer readable instructions are further configured to receive data indicating patient details and to configure said displayed graphic in response to said received data.

30. A device according to claim 29, wherein said graphic is provided as part of a game, and wherein said game is configured based upon said received data.

31. A device according to claim 30, wherein said received data indicates a required number of cycles and said game is configured to provide a number of levels equal to said required number of cycles.

32. A device according to claim 31, wherein said received data indicates a required number of inhalations per cycle equal and to provide a number of stages for each level equal to said required number of inhalations.

33. A device according to claim 29, wherein said memory comprises a plurality of games and said computer readable instructions are configured to select one of said plurality of games based on received details.

34. A device according to claim 30, wherein said received data comprises data indicating an age of said patient and a difficulty level of said game is configured based upon said indicated age.

35. A device according to claim 24, wherein said computer readable instructions are further configured to, if an indication of a successful inhalation is not detected by said detection means within a predetermined time period, cause a prompt to be presented on said display to incentivise said user to complete a successful inhalation of said medicament.

36. A device according to claim 25, wherein said detection means are movable in relation to said outlet, and said computer readable instructions are configured to perform a calibration operation comprising:
receiving from said detection means a first calibration pressure;
determining whether said first calibration pressure satisfies a predetermined criterion; and
displaying a prompt on said display for said detection means to be moved in relation to said outlet if said first calibration pressure does not satisfy said predetermined criterion.

37. A device according to claim 24, wherein said incentive graphic is adapted to indicate progress towards completion of said dosage regime.

38. A device according to claim 37, wherein said incentive graphic is adapted to indicate an endpoint of said progress and a current progress towards said endpoint.

39. A computer implemented method for improving compliance with a medicament dosage regime, said regime requiring inhalation of a predetermined dosage of a medicament using an inhaler spacer, the method comprising:
displaying at least one incentive graphic on a display device;
detecting a successful inhalation of a user of the inhaler spacer; and
in response to detecting said successful inhalation, updating said incentive graphic.

40. A non-transitory computer-readable storage medium storing one or more programs for improving compliance with a medicament dosage regime, said regime requiring inhalation of a predetermined dosage of a medicament using an inhaler spacer, the one or more programs for execution by one or more processors of a computer system, the one or more programs comprising instructions for:
displaying at least one incentive graphic on a display device;
detecting a successful inhalation of a user of the inhaler spacer; and
in response to detecting said successful inhalation, updating said incentive graphic.

41. The non-transitory computer-readable storage medium of claim 40, wherein said detecting the successful inhalations comprise exhalations through an outlet of said inhaler spacer.

42. A device mount for use with an inhaler spacer, comprising:
support means configured to support a device according to claim 24.

43. An inhaler spacer device, comprising:
a mouthpiece;
a spacer configured to connect the mouthpiece to a medicament delivery device, said spacer having an air outlet; and
an incentive device comprising:
detection means for detecting indications of a successful inhalation of by a user of the inhaler device;
a display; and
a memory storing computer readable instructions configured to receive indications of successful inhalations detected by said detection means and to provide an incentive graphic on said display, said incentive graphic being updated in response to received indications of successful inhalations to indicate progress towards completion of a predetermined dosage requirement.
